(19) United States
(12) Patent Application Publication
(10) Pub. No.: US 2024/0082222 A1
(43) Pub. Date: Mar. 14, 2024

(54) 4-CHLORO-N-[2-[(4-CHLOROPHENYL)METHYL]-3-OXO-1,2,4-THIADIAZOL-5-YL]BENZAMIDE FOR USE IN MEDICINE

(71) Applicant: Betagenon AB, Umeå (SE)

(72) Inventors: Thomas EDLUND, Umeå (SE); Jacob WESTMAN, Järlåsa (SE)

(73) Assignee: Betagenon AB, Umeå (SE)

(21) Appl. No.: 18/271,627

(22) PCT Filed: Jan. 11, 2022
(86) PCT No.: PCT/GB2022/050054

§ 371 Date: Jul. 10, 2023

(30) Foreign Application Priority Data
Jan. 12, 2021 (GB) 2100352.0

(51) Int. Cl. A61K 31/433; A61K 9/00; A61K 9/20

(52) U.S. Cl. CPC A61K 31/433; A61K 9/0053; A61K 9/2009; A61K 9/2013; A61K 9/2018; A61K 9/2054; A61K 9/2059

(57) ABSTRACT

This invention relates to a new method of activating 5′ adenosine monophosphate-activated protein kinase (AMPK) to treat certain diseases and disorders using a salt of 4-chloro-N[2-[(4-chlorophenyl)methyl]-3-oxo-1,2,4-thiadiazol-5-yl]benzamide in a dose-efficient manner. Diseases that may be treated in this way include type-2 diabetes.

### FIELD OF THE INVENTION

The present invention relates to the use of a sodium salt of a particular pharmaceutical active ingredient in medicine in a dose-efficient manner by activating 5′ adenosine monophosphate-activated protein kinase (AMPK) and thereby treating particular diseases.

### BACKGROUND OF THE INVENTION

AMP-activated protein kinase (AMPK) is a protein kinase enzyme that consists of three protein sub-units and is activated by hormones, cytokines, exercise, and stresses that diminish cellular energy state (e.g. glucose deprivation). Activation of AMPK increases processes that generate adenosine 5′-triphosphate (ATP) (e.g., fatty-acid oxidation) and restrains others such as fatty acid-, glycerolipid- and protein-synthesis that consume ATP, but are not acutely necessary for survival. Conversely, when cells are presented with a sustained excess of glucose, AMPK activity diminishes and fatty acid, alycerolipid- and protein-synthesis are enhanced. AMPK thus is a protein kinase enzyme that plays an important role in cellular energy homeostasis, Therefore, the activation of AMPK is coupled to glucose lowering effects and triggers several other biological effects, including the inhibition of cholesterol synthesis, lipogenesis, triglyceride synthesis, and the reduction of hyperinsulinemia.

Given the above, AMPK is a preferred target for the treatment of the metabolic syndrome and especially type 2 diabetes. AMPK is also involved in a number of pathways that are important for many different diseases (e.g. AMPK is also involved in a number of pathways that are important in CNS disorders, inflammation (and resultant fibrosis), osteoporosis, heart failure and sexual dysfunction).

AMPK is also involved in a number of pathways that are important in cancer. Several tumour suppressors are part of the AMPK pathway. AMPK acts as a negative regulator of the mammalian TOR (mTOR) and EF2 pathway, which are key regulators of cell Growth and proliferation. The deregulation may therefore be linked to diseases such as cancer (as well as diabetes). AMPK activators may therefore be of utility as anti-cancer drugs.

The effects of the dysregulation of AMPK in diseases such as obesity, inflammation, diabetes and cancer are discussed in, for example, Jeon S-M, Experimental & Molecular Medicine (2016) 48, e245.

It has been shown that AMPK activator drugs (e.g. metformin and 4-chloro-N-[2-[(4-chlorophenyl)methyl]-3-oxo-1,2,4-thiadiazol-5-yl]benzamide (i.e. the compound of formula I below)) are effective at treating pain. Das and co-workers report that, following lumbar disc puncture, postinjury treatment in mice with AMPK activator drugs reduces mechanical hypersensitivity (Das V, et al, Reg Anesth Pain Med 2019;0:1-5. doi:10.1136/rapm-2019-100839). Similarly, Das and co-workers also report that early treatment with AMPK activator drugs reduces mechanical hypersensitivity in a postoperative pain model in mice (Das V, et al. Reg Anesth Pain Med 2019;0:1.-6, doi:10.1136/rapm-2019-100651). These drugs also normalize the AMPK pathway in the dorsal root Ganglion. AMPK activators may therefore be used in the treatment of pain, particularly post-operative pain.

It has also been shown that hepatic steatosis may be regulated by AMPK (Zhao et al. J. Biol. Chem. 2020 295: 12279-12289). Activation of AMPK inhibits de novo lipogenesis while promoting fatty acid oxidation (β-oxidation) in the liver. AMPK activation also reduces free fatty acid release from adipose tissue and prevents hepatic steatosis. Pharmacological activation of AMPK in the liver was reported to promote beneficial effects on multiple aspects of non-alcoholic fatty liver disease (NAFLD). For example, activation of AMPK was found to improve non-alcoholic steatohepatitis (NASH) in both murine and simian animal models. Accordingly, AMPK activators may be useful in the treatment of NAFLD and NASH.

An example of an AMPK activator is 4-chloro-N-[2-[(4-chlorophenyl)methyl]-3-oxo-1,2,4-thiadiazol-5-yl]benzamide (i.e., the compound of formula I), which was first disclosed in WO 2011/004162.

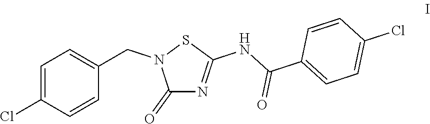 [SMILES: O=C(Nc1nc(=O)n(Cc2ccc(Cl)cc2)s1)c1ccc(Cl)cc1]

As an AMPK agonist (i.e. an AMPK activator), the compound of formula I is useful in the treatment of disorders or conditions which are ameliorated by the activation of AMPK. Such compounds may be useful in the treatment of cardiovascular disease (such as heart failure), diabetic kidney disease, type 2 diabetes, insulin resistance, non-alcoholic fatty liver disease, non-alcoholic steatohepatitis, pain, opioid addiction, obesity, cancer, inflammation (including chronic inflammatory diseases), autoimmune diseases, osteoporosis and intestinal diseases.

There remains a need to improve the bioavailability of active ingredients, such as 4-chloro-N-[2-[(4-chlorophenyl)methyl]-3-oxo-1,2,4-thiadiazol-5-yl]benzamide, in vivo so as to improve their effectiveness in medicine. The inventors have now found a treatment that surprisingly enhances the bioavailability of the compound of formula I in vivo.

The listing or discussion of an apparently prior-published document in this specification should not necessarily be taken as an acknowledgement that the document is part of the state of the art or is common general knowledge.

### DETAILED DESCRIPTION OF THE INVENTION

According to a first aspect of the invention, there is provided a method of activating 5′ adenosine monophosphate-activated protein kinase (AMPK) comprising administering from about 200 to about 1000 mg/day of a sodium salt of 4-chloro-N-[2-[(4-chlorophenyl)methyl]-3-oxo-1,2,4-thiadiazol-5-yl]benzamide in a pharmaceutical dosage form to a human subject.

Methods according to the first aspect of the invention are hereinafter referred to as “methods of the invention”.

According to an alternative first aspect of the invention, there is provided from about 200 to about 1000 mg/day of a sodium salt of 4-chloro-N-[2-[(4-chlorophenyl)methyl]-3-oxo-1,2,4-thiadiazol-5-yl]benzamide in a pharmaceutical dosage form for use in activating AMPK.

According to a further alternative first aspect of the invention, there is provided the use of from about 200 to about 1000 mg/day of a sodium salt of 4-chloro-N-[2-[(4-chlorophenyl)methyl]-3-oxo-1,2,4-thiadiazol-5-yl]benzamide in a pharmaceutical dosage form, in the manufacture of a medicament for treating a disease or disorder by activating AMPK.

It will be understood that a “sodium salt” is a chemical compound consisting of an assembly of cations of sodium and associated anions. Accordingly, the term “a sodium salt of 4-chloro-N-[2-[(4-chlorophenyl)methyl]-3-oxo-1,2,4-thiadiazol-5-yl]benzamide” refers to a compound comprising sodium cations and anions of 4-chloro-N-[2-[(4-chlorophenyl)methyl]-3-oxo-1,2,4-thiadiazol-5-yl]benzamide. For example, a sodium salt of 4-chloro-N-[2-[(4-chlorophenyl)methyl]-3-oxo-1,2,4-thiadiazol-5-yl]benzamide may refer to the compound of formula II,

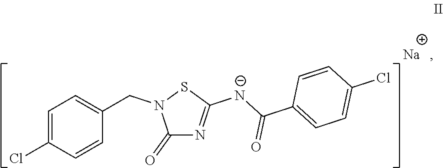

wherein Na⁺ represents the sodium cation.

The skilled person will recognize that, when dissolved in a suitable solvent (e.g. water), the sodium salt of 4-chloro-N-[2-[(4-chlorophenyl)methyl]-3-oxo-1,2,4-thiadiazol-5-yl]benzamide may dissociate into its anionic and cationic components.

Throughout this specification, structures may or may not be presented with chemical names. Where any question arises as to nomenclature, the structure prevails. Where it is possible for the compound to exist as a tautomer (e.g. in an alternative resonance form) the depicted structure represents one of the possible tautomeric forms, wherein the actual tautomeric form(s) observed may vary depending on environmental factors such as solvent, temperature or pH. All tautomeric (and resonance) forms and mixtures thereof are included within the scope of the invention. For example, the following tautomers are included within the scope of the invention:

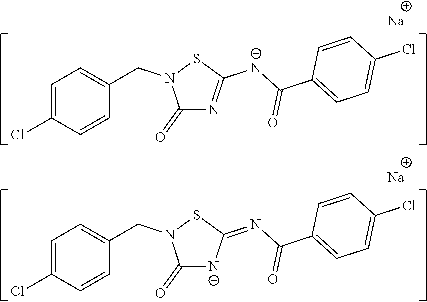

For the avoidance of doubt, sodium salts of 4-chloro-N-[2-[(4-chlorophenyl)methyl]-3-oxo-1,2,4-thiadiazol-5-yl]benzamide are solid under ambient conditions, and references herein to said salts include all amorphous, crystalline and part crystalline forms thereof.

Sodium salts of 4-chloro-N-[2-[(4-chlorophenyl)methyl]-3-oxo-1,2,4-thiadiazol-5-yl]benzamide may be prepared in accordance with techniques that are well known to those skilled in the art. For example, 4-chloro-N-[2-[(4-chlorophenyl)methyl]-3-oxo-1,2,4-thiadiazol-5-yl]benzamide may be reacted with sodium hydroxide, or an alternative sodium base compound. Salt switching techniques may also be used to convert one salt into another salt.

Where the salt is prepared from 4-chloro-N-[2-[(4-chlorophenyl)methyl]-3-oxo-1,2,4-thiadiazol-5-yl]benzamide, 4-chloro-N-[2-[(4-chlorophenyl)methyl]-3-oxo-1,2,4-thiadiazol-5-yl]benzamide may be prepared in accordance with techniques that are well known to those skilled in the art, such as those described in international patent application WO 2011/004162. The contents of WO 2011/004162 are incorporated by reference.

The sodium salt of 4-chloro-N-[2-[(4-chlorophenyl)methyl]-3-oxo-1,2,4-thiadiazol-5-yl]benzamide is referred to herein as “the salt of the invention”.

The method of the invention has been found to be surprisingly effective at improving (e.g. increasing) the bioavailability of 4-chloro-N-[2-[(4-chlorophenyl)methyl]-3-oxo-1,2,4-thiadiazol-5-yl]benzamide in vivo compared to a method comprising administration of a comparable amount of 4-chloro-N-[2-[(4-chlorophenyl)methyl]-3-oxo-1,2,4-thiadiazol-5-yl]benzamide in the free base form. Improvement in bioavailability may be demonstrated by measuring the Cₘₐₓ or the area under the curve (AUC) following administration of the pharmaceutical dosage form to a human subject. It has also been found that a comparable level of systemic exposure to 4-chloro-N-[2-[(4-chlorophenyl)methyl]-3-oxo-1,2,4-thiadiazol-5-yl]benzamide, e.g. a comparable concentration thereof in blood plasma, in a human subject can be achieved through administration of a significantly lower amount of 4-chloro-N-[2-[(4-chlorophenyl)methyl]-3-oxo-1,2,4-thiadiazol-5-yl]benzamide in the form of a sodium salt of that compound. The finding that administration of the sodium salt form can enable a reduction in the dose required to achieve a particular level of systemic exposure is beneficial as use in a dose-efficient manner reduces the likelihood of unwanted side effects occurring. The salt of the invention, and pharmaceutical dosage form comprising said salt, is useful in the therapies described herein in a subject in need of such therapy.

In the context of the present invention, the term “free base” refers to a form of 4-chloro-N-[2-[(4-chlorophenyl)methyl]-3-oxo-1,2,4-thiadiazol-5-yl]benzamide which is not in a salt form. Free base 4-chloro-N-[2-[(4-chlorophenyl)methyl]-3-oxo-1,2,4-thiadiazol-5-yl]benzamide may be depicted as the compound of formula I,

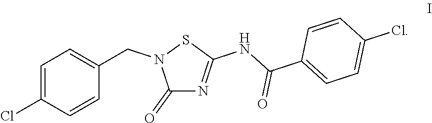 [SMILES: O=C(Nc1nc(=O)n(Cc2ccc(Cl)cc2)s1)c1ccc(Cl)cc1]

The terms “Cₘₐₓ” and “AUC” will be well understood by the person skilled in the art to refer, in the present context, to the peak plasma concentration of 4-chloro-N-[2-[(4-chlorophenyl)methyl]-3-oxo-1,2,4-thiadiazol-5-yl]benzamide after administration (e.g. to a human subject) and the integral of the concentration/time curve for that substance following the administration of the salt of the invention in a pharmaceutical dosage form, respectively.

Thus, the method of the invention is capable of increasing the bioavailability of the compound of formula I in humans compared to a method comprising administration of the free base form of said compound. By this, we mean that administration of a pharmaceutical dosage form comprising the salt of the invention results in a larger systemically available fraction of 4-chloro-N-[2-[(4-chlorophenyl)methyl]-3-oxo-1,2,4-thiadiazol-5-yl]benzamide in vivo compared to administration of a pharmaceutical dosage form comprising the free base form of said compound. The increase in the amount of the compound of formula that is systemically available following administration of a pharmaceutical dosage form comprising the salt of the invention as compared to administration of a pharmaceutical dosage form comprising the free base form of said compound may be at least about 10%, (at least) about 20%, about 30%, about 40%, about 50%, about 60%, about 70%, about 80%, about 90%, about 100% (i.e. 2-fold), about 150%, about 200% (i.e. 3-fold), about 250%, about 300% (i.e. 4-fold), about 350%, or about 400% (i.e. 5-fold).

The improvement in the bioavailability at a given dose of the salt, or the achievement of comparable systemic exposure through administration of a reduced dose of the salt (compared to the dose of the non-salt form required to achieve that exposure), may be demonstrated using suitable methods known in the art. For example, changes in the bioavailability and systemic exposure levels may be observed by comparing the pharmacokinetic data (e.g. Cₘₐₓ data) for a subject who has been administered a pharmaceutical dosage form comprising the salt of the invention with the corresponding data for a subject who has been administered a pharmaceutical dosage form comprising 4-chloro-N-[2-[(4-chlorophenyl)methyl]-3-oxo-1,2,4-thiadiazol-5-1,2,4-thiadiazol-5-yl]benzamide in the free base form.

The skilled person will understand that the method of the invention comprises administering a total dosage of from about 200 to about 1000 mg per day (mg/day) of the sodium salt of 4-chloro-N-[2-[(4-chlorophenyl)methyl]-3-oxo-1,2,4-thiadiazol-5-yl]benzamide by way of one or more pharmaceutical dosage forms described herein. In particular embodiments, the total dosage of the sodium salt of 4-chloro-N-[2-[(4-chlorophenyl)methyl]-3-oxo-1/2/4-thiadiazol-5-yl]benzamide administered to the human subject may be in the range of from about 200 to about 800 mg/day, about 200 to about 600 mg/day, or, preferably, about 200 to about 400 mg/day.

Advantageously, the salt of the invention (including pharmaceutical dosage forms comprising said salt) may be administered to the human subject in a single daily dose (e.g. via oral delivery). Alternatively, the total daily dosage of the salt of the invention may be administered in divided doses two, three or four times daily (e.g. twice daily with reference to the doses described herein, such as a dose of 100 mg, 250 mg, or 500 mg twice daily). Still further, the method of the invention may involve administration at a frequency of less than once daily, e.g., once every two days, once weekly or twice weekly. In such embodiments, the average daily dose received by the subject will still be from about 200 to about 1000 mg. In a particular embodiment, the salt of the invention is administered not more than once per day. More particularly, the salt of the invention is administered in once daily.

As indicated by Example 3, the method of the invention is particularly effective when the salt of the invention is administered once daily for a duration of at least one week (e.g. at least 7, 8, 9, 10, 11, 12, 13, 14, 15, 16, 17, 18, 19, 20, 21, 22, 23, 24, 25, 26, 27 or 28 days). In particular embodiments, the duration is at least two weeks. In further embodiments, the duration of administration is at least three weeks. In other embodiments, the salt of the invention is administered once daily for a duration that is at least sufficient to achieve a steady state blood plasma concentration of 4-chloro-N-[2-[(4-chlorophenyl)methyl]-3-oxo-1,2,4-thiadiazol-5-yl]benzamide. Longer periods of treatment are envisaged, including treatment that may extend over many months or years, as is deemed appropriate by a prescribing doctor under the circumstances. It is intended that such extended treatments are also the methods of the invention.

The term “about” as used herein when referring to a measurable value such as an amount of a compound, dose, time, temperature, and the like, refers to variations of 20%, 10%, 5%, 1%, 0.5%, or even 0.1% of the specified amount. It is contemplated that, at each instance, such terms may be replaced with the notation “±10%”, or the like (or by indicating a variance of a specific amount calculated based on the relevant value). It is also contemplated that, at each instance, such terms may be deleted.

For the avoidance of doubt, the dose administered to a human subject, in the context of the present invention, should be sufficient to activate AMPK and thereby effect a therapeutic response in the subject over a reasonable timeframe. One skilled in the art will recognize that the selection of the exact dose and composition and the most appropriate delivery regimen will also be influenced by inter alia the pharmacological properties of the dosage form, the nature and severity of the condition being treated, and the physical condition and mental acuity of the recipient, as well as the potency of the specific compound, the age, condition, body weight, sex and response of the subject to be treated, and the stage/severity of the disease.

In any event, the medical practitioner, or other skilled person, will be able to determine routinely the actual dosage which will be most suitable for an individual subject.

The method of the invention may be particularly advantageous in that it enables a clinician to achieve a desired peak blood plasma concentration of 4-chloro-N-[2-[(4-chlorophenyl)methyl]-3-oxo-1,2,4-thiadiazol-5-yl]benzamide in a subject whilst administering a lower dose of the active ingredient to that subject. In the study described in Example 2, repeated dosing of the sodium salt of 4-chloro-N-[2-[(4-chlorophenyl)methyl]-3-oxo-1,2,4-thiadiazol-5-yl]benzamide was shown to result in a Cmax of around 50 μg/ml. In a prior study involving administration of 4-chloro-N-[2-[(4-chlorophenyl)methyl]-3-oxo-1,2,4-thiadiazol-5-yl]benzamide in suspension, a similar Cmax was achieved only when subjects received a substantially larger (ca. five-fold larger) repeat dose amount.

In addition, as shown by the study described in Example 3, it is possible to achieve a peak blood plasma concentration of 4-chloro-N-[2-[(4-chlorophenyl)methyl]-3-oxo-1,2,4-thiadiazol-5-yl]benzamide of at least around 85 μg/ml following repeated dosing of 212 mg the salt of the invention in the form of a tablet and at least around 50 μg/ml following repeated dosing of 200 mg the salt of the invention in the form of a capsule.

The method of the invention is therefore capable of achieving a peak blood plasma concentration of 4-chloro-N-[2-[(4-chlorophenyl)methyl]-3-oxo-1,2,4-thiadiazol-5-yl]benzamide of at least 40 μg/mL (e.g. after repeated dosing of 200 mg the salt of the invention daily for at least two weeks). In further embodiments, method of the invention achieves a peak blood plasma concentration of at least 50, 60, 70, 80, 90, 100, 110, 120 or 130 μg/mL.

The peak blood plasma concentration may be arrived at following administration of a sufficient number of doses to achieve a steady state blood plasma concentration, or to achieve a blood plasma concentration profile approaching the steady state profile. In this context, a steady state concentration is achieved when the variation in the concentration of analyte (in this case 4-chloro-N-[2-[(4-chlorophenyl)methyl]-3-oxo-1,2,4-thiadiazol-5-yl]benzamide) in the blood plasma remains within clinically acceptable bounds over the period between successive dosages of the salt of the invention. A steady state concentration may also be considered to be achieved when the variation in the Cₘₐₓ also remains within clinically acceptable bounds following consecutive administrations. Therefore, in one embodiment, the peak blood plasma concentration of 4-chloro-N-[2-[(4-chlorophenyl)methyl]-3-oxo-1,2,4-thiadiazol-5-yl]benzamide is reached after achieving a steady state concentration.

The time required to arrive at the steady state will vary between subjects. The steady state for a drug is typically reached after 4 to 5 half-lives (t^{1/2}) of the drug have passed following administration. The skilled person (i.e. a clinician) will be able to appreciate when the steady state has been achieved by reference to clinical evaluations of the subject's blood, e.g. using methods referred to in Example 2 and Example 3. Typically, the steady state concentration of 4-chloro-N-[2-[(4-chlorophenyl)methyl]-3-oxo-1,2,4-thiadiazol-5-yl]benzamide is obtained after repeated dosing has taken place for around two weeks, though a longer time may be required. For example, the peak blood plasma concentration may be reached after 15, 16, 17 or 18 days.

Unless indicated otherwise, all technical and scientific terms used herein will have their common meaning as understood by one of ordinary skill in the art to which this invention pertains.

For the avoidance of doubt, the skilled person will understand that references herein to particular aspects of the invention (such as the first aspect of the invention) will include references to all embodiments and particular features thereof, which embodiments and particular features may be taken in combination to form further embodiments and features of the invention.

### Pharmaceutical Dosage Forms

As indicated herein, the salt of the invention are useful as therapeutic agents for activating AMPK and thereby treating a variety of medical disorders and conditions. The salt of the invention is administered to a human subject in need thereof in the form of a pharmaceutical formulation, which is also referred to herein as a pharmaceutical dosage form.

In an embodiment, the salt of the invention is the sole active pharmaceutical ingredient present in the dosage form. In a further embodiment, the salt of the invention (or a pharmaceutically acceptable salt or solvate thereof) is present in the dosage form alongside one or more other active pharmaceutical ingredients, or may be administered as part of a combination therapy with one or more other active pharmaceutical ingredients.

In particular embodiments, the method comprises administration of pharmaceutical dosage form of the salt of the invention, including all embodiments and particular features thereof, wherein said salt is provided in the form of particles having a particle size distribution defined by a D90 of less than about 10 μm (e.g. as measured using laser diffraction). Particle sizes are typically reduced by milling larger particles of a given substance.

The term “milling” (which may be used interchangeably with other terms of the art such as “reducing size”, “comminuting”, “grinding”, and “pulverising”), as used herein, refers to the process of subjecting a solid sample (e.g. granules) to mechanical energy to reduce the particle size of the solid sample. For example, coarse particles may be broken down to finer ones, such that the average particle size is reduced to meet desired parameters.

Milling is regarded as a ‘top-down’ approach to the production of fine particles. For example, a drug solid may be cut by sharp blades (e.g. cutter mill), impacted by hammers, subjected to high pressure homogenisation, or crushed or compressed by the application of pressure (e.g. roller-mill or pestle and mortar). As a limited amount of energy is typically imparted, particles produced by such methods remain relatively coarse. Technological advancements in milling equipment have enabled the production of ultrafine drug particles down to micron (i.e. the μm unit range) or even sub-micron (e.g. the nm unit range) dimensions.

Certain milling processes may be characterised as being dry milling processes. Such processes are preferred for processing of the salts of the invention.

‘Dry milling’ refers to a process in which a drug is milled in its dry state, i.e. in the absence of a liquid medium (e.g. in the substantial absence of water). In the dry state, the drug can be milled alone, or in the presence of one or more other components, such as pharmaceutically acceptable excipients. Other abrasive materials, such as salts, may be present during the milling process to aid in the particle size reduction. The mechanical energy imparted by dry milling fosters interactions between particles of the drug (and optionally other substances present) via van der Waals forces or hydrogen bonding.

A review of milling processes for pharmaceutical products may be found in e.g. Loh et al., Asian Journal of Pharmaceutical Sciences, 10 (2015), 255-274. Excipients suitable for inclusion in drug particles are known in the art, e.g. as described in Peltonen et al., in Handbook of Polymers for Pharmaceutical Technologies, ed. Thakur and Thakur, Wiley, volume 4, chapter 3, 67-87, and Nekkanti et al, in Drug Nanoparticles—An Overview, The Delivery of Nanoparticles, IntechOpen. The content of these documents is incorporated by reference.

Stabilisers, such as polymers and surfactants, are often used during milling processes in order to increase the repulsion between particles and inhibit aggregation. Aggregation of finely ground particles may occur during micronisation, which can ultimately slow down the dissolution process and affect bioavailability. The increase in the systemic exposure of 4-chloro-N-[2-[(4-chlorophenyl)methyl]-3-oxo-1,2,4-thiadiazol-5-yl]benzamide has been found to occur following administration of dry milled salt of the active ingredient even without the addition of stabilisers. Thus, in one embodiment, the pharmaceutical formulation does not comprise any stabilisers.

Milling reduces the average size of the particles containing the sodium salt of 4-chloro-N-[2-[(4-chlorophenyl)methyl]-3-oxo-1,2,4-thiadiazol-5-yl]benzamide. The extent and effectiveness of the milling may be determined by measuring the particle size distribution of said particles before and after the milling process by any suitable method. The term “particle size distribution” refers to the relative number of particles present according to size in a solid sample, such as a powder, a granular material, or particles dispersed in a fluid.

The particle size distribution of a solid sample may be measured using techniques that are well known in the art. For example, the particle size distribution of a solid sample may be measured by laser diffraction, dynamic light scattering, image analysis (e.g. dynamic image analysis), sieve analysis, air elutriation analysis, optical counting, electro-resistance counting, sedimentation, laser obscuration and acoustic (e.g. ultrasound attenuation) spectroscopy. Particular methods that may be mentioned for measuring the particle size distribution of particles of the salt of the invention are dynamic light scattering and laser diffraction.

Particle size distributions may be also determined based on results from sieve analysis. Sieve analysis presents particle size information in the form of an S-curve of cumulative mass retained on each sieve versus the sieve mesh size. The most commonly used metrics when describing particle size distributions are D-values (e.g. D10, D50 and D90, which are the intercepts for 10%, 50% and 90% of the cumulative mass, respectively). The particle size distribution of the present invention is preferably defined using one or more of such values. D-values essentially represent the diameter of the sphere which divides the sample's mass into a specified percentage when the particles are arranged on an ascending mass basis. For example, the D10 value is the diameter at which 10% of the sample's mass is comprised of particles with a diameter of less than this value. The D50 value is the diameter of the particle that 50% of a sample's mass is smaller than and 50% of a sample's mass is larger than.

In one embodiment, the particles containing a salt of the invention may have a particle size distribution defined by a D90 of less than about 10 μm (e.g. from about 5 μm to about 10 μm) (e.g. as measured using laser diffraction). The particle size distribution may alternatively be defined by a D90 of less than about 8 μm (e.g. from about 5 μm to about 8 μm). In a further embodiment, the particles consisting of the salt of the invention may have a particle size distribution defined by a D50 of less than about 6 μm (e.g. from about 0.5 μm to about 6 μm). In a yet further embodiment, the particle size distribution of the particles consisting of the salt of the invention may further be a defined by a D10 of less than about 2 μm (e.g. from about 0.2 μm to about 2 μm).

The particle size distribution parameters mentioned above may be applicable, individually or in combination. For example, in particular embodiments, the dosage form comprises particles containing the salt of the invention, said particles having a particle size distribution defined by a D90 of less than about 10 μm and a D50 of less than about 6 μm. Still further, said particles may have a particle size distribution defined by a D90 of less than 9 μm; a D50 of less than 6 μm or less than 5 μm; and a D10 of less than 2 μm or less than 1.5 μm.

The particle size distribution of particles containing the salt of the invention may be measured by laser diffraction, using, for example a commercially available particle size analyser such as a Malvern Instrument, Mastersizer 3000.

The present invention also encompasses a pharmaceutical dosage form comprising particles containing the salt of the invention with any of the particle size distributions defined herein, regardless of the process by which the particles are produced.

Preferably, the pharmaceutical dosage form comprises particles of the salt of the invention with any of the particular particle size distributions described herein, wherein the particles are obtained by a process which involves milling said salt.

The skilled person will understand that the pharmaceutical dosage forms described herein may act systemically, and may therefore be administered accordingly using suitable techniques known to those skilled in the art. The pharmaceutical dosage form as described herein will normally be administered orally, i.e. as an oral pharmaceutical dosage form. Thus, in a second aspect of the invention, there is provided an oral pharmaceutical dosage form comprising from about 200 to about 1000 mg of a sodium salt of 4-chloro-N-[2-[(4-chlorophenyl)methyl]-3-oxo-1,2,4-thiadiazol-5-yl]benzamide.

In particular embodiments, the pharmaceutical dosage form referred to in the first and second aspects of the invention may comprise, for example, from about 200 mg to about 800 mg, from about 200 mg to about 600 mg, or from about 200 mg to about 400 mg) of the salt of the invention. Preferably, the pharmaceutical dosage form of the comprises from about 200 to about 400 mg of the sodium salt of 4-chloro-N-[2-[(4-chlorophenyl)methyl]-3-oxo-1,2,4-thiadiazol-5-yl]benzamide.

Dosage forms intended for oral administration may further comprise an enteric coating in order to prevent or minimise dissolution or disintegration in the gastric environment. As such, oral preparations (e.g. capsules or tablets) coated by an enteric coating may provide targeted release of the salt of the invention in the small intestine. For example, the enteric coating may be present on surface of the formulation (e.g. on the surface of a tablet or a capsule), or each of the particles containing the salt of the invention may be coated with the enteric coating. Thus, in particular embodiments, the pharmaceutical dosage form used in the method of the invention further comprises an enteric coating.

It may be desirable to minimise dissolution or disintegration of an oral pharmaceutical dosage form (e.g. a capsule or a tablet, and the like) in the gastric environment and/or provide targeted release of the active ingredient in the small intestine. Thus, in particular embodiments, the enteric coating is present on the pharmaceutical dosage form of the method of the invention. For example, said coating may be provided as an outer layer on the pharmaceutical dosage form.

Alternatively, particles containing the salt of the invention may be individually coated with the enteric coating, and said coated particles may be prepared into the pharmaceutical dosage form. Thus, in particular embodiments, the pharmaceutical dosage form contains particles comprising the salt of the invention and each particle is coated with the enteric coating.

The term “enteric coating” refers to a substance (e.g. a polymer) that is incorporated into an oral medication (e.g. applied onto the surface of a tablet, a capsule, particles or pellets) and that inhibits dissolution or disintegration of the medication in the gastric environment. Enteric coatings are typically stable at the highly acidic pH found in the stomach, but break down rapidly in the relatively basic pH of the small intestine. Therefore, enteric coatings prevent release of the active ingredient in the medication until it reaches the small intestine.

Any enteric coating known to the skilled person may be used in the present invention. Particular enteric coating materials that may be mentioned include those which comprise beeswax, shellac, an alkylcellulose polymer resin (e.g. ethylcellulose polymers, carboxymethylethylcellulose, or hydroxypropyl methylcellulose phthalate) or an acrylic polymer resin (e.g. acrylic acid and methacrylic acid copolymers, methyl methacrylate copolymers, ethoxyethyl methacrylates, cyanoethyl methacrylate, methacrylate copolymers, methacrylic acid copolymer, aminoalkyl methacrylate copolymer, poly(acrylic acid), poly(methacrylic acid), methacrylic acid alkylamide copolymer, poly(methyl methacrylate), poly(methacrylic acid) (anhydride), polymethacrylate, methyl methacrylate copolymer, poly(methyl methacrylate) copolymer, polyacrylamide, poly(methacrylic acid anhydride), and glycidyl methacrylate copolymers), cellulose acetate phthalate and polyvinyl acetate phthalate.

The pharmaceutical dosage forms referred to in the first and second aspects of the invention may be provided in the form of a tablet or particularly a capsule. For example, capsules such as soft gelatin capsules may be prepared containing the salt of the invention alone, or together with a suitable vehicle, e.g. vegetable oil, fat etc.

Similarly, hard gelatin capsules may contain the salt of the invention alone, or in combination with solid powdered ingredients such as a disaccharide (e.g. lactose or saccharose), an alcohol sugar (e.g. sorbitol or mannitol), a vegetable starch (e.g. potato starch or corn starch), a polysaccharide (e.g. amylopectin or cellulose derivatives) or gelling agent (e.g. gelatin).

The pharmaceutical dosage forms described herein may be prepared in accordance with standard and/or accepted pharmaceutical practice. The pharmaceutical dosage forms of the first and second aspects of the invention will generally be provided as a mixture comprising the salt of the invention and one or more pharmaceutically acceptable excipients. The one or more pharmaceutically acceptable excipients may be selected with due regard to the intended route of administration in accordance with standard pharmaceutical practice. Such pharmaceutically acceptable excipients are preferably chemically inert to the active compound and are preferably have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). A brief review of methods of drug delivery may also be found in e.g. Langer, Science 249, 1527 (1990).

Thus, according to particular embodiments, the pharmaceutical dosage forms referred to in the first and second aspects of the invention further comprises at least one pharmaceutically acceptable excipient. In particular, the at least one pharmaceutically acceptable excipient may be a lubricant, a binder, a filler, a surfactant, a diluent, an anti-adherent, a coating, a flavouring, a colourant, a glidant, a preservative, a sweetener, a disintegrant, an adsorbent, a buffering agent, an antioxidant, a chelating agent, a dissolution enhancer, a dissolution retardant or a wetting agent.

Particular pharmaceutically acceptable excipients that may be mentioned include mannitol, PVP (polyvinylpyrrolidone) K30, lactose, saccharose, sorbitol, starch, amylopectin, cellulose derivatives, gelatin, or another suitable ingredients, as well as disintegrating agents and lubricating agents such as sodium lauryl sulfate, Na-docusate, magnesium stearate, calcium stearate, sodium stearyl fumarate and polyethylene glycol waxes. In the preparation of a pharmaceutical dosage form of the salt of the invention for oral administration, particles containing the salt of the invention (preferably milled) may be mixed, either together or separately, with mannitol, PVP (polyvinylpyrrolidone) K30 and sodium lauryl sulfate.

In the preparation of a pharmaceutical dosage form for use in the method of the invention, the salt of the invention may be mixed, either together or separately, with one or more of the pharmaceutical excipients (including basic excipients) listed above.

Mixtures of the salt of the invention and one or more pharmaceutically acceptable excipients may be processed into pellets or granules, or compressed into tablets. Thus, pharmaceutical dosage form of the method of the inventions may be a tablet, mini-tablets, blocks, pellets, particles, granules or a powder for oral administration.

### Medical Uses

The dose-efficient methods of activating AMPK and the pharmaceutical dosage forms described herein are useful in medicine. 4-Chloro-N-[2-[(4-chlorophenyl)methyl]-3-oxo-1,2,4-thiadiazol-5-yl]benzamide is a known AMPK activator, and AMPK activation is known to be beneficial in the treatment of a variety of disease, as disclosed in international patent application nos. WO 2011/004162 and WO 2020/095010.

Thus, according to a third aspect of the invention, there is provided the use of an oral pharmaceutical dosage form according to the second aspect of the invention in the manufacture of a medicament for the treatment of a disease or disorder by activating AMPK, wherein from about 200 to about 1000 mg/day of the sodium salt of 4-chloro-N-[2-[(4-chlorophenyl)methyl]-3-oxo-1,2,4-thiadiazol-5-yl]benzamide is administered to a human subject.

Similarly, the method of activating AMPK according to the first aspect of the invention may be performed to treat a disease or disorder ameliorated by AMPK activation in the human subject.

By ‘activate AMPK’, we mean that the steady state level of phosphorylation of the Thr-172 moiety of the AMPK-α (AMPK-alpha) subunit is increased compared to the steady state level of phosphorylation in the absence of a compound of formula I. Alternatively, or in addition, we mean that there is a higher steady state level of phosphorylation of any other proteins downstream of AMPK, such as acetyl-CoA carboxylase (ACC).

Diseases or disorders that are treated by activating AMPK will be known by those skilled in the art to include cardiovascular disease (such as heart failure, e.g. heart failure with preserved ejection fraction), diabetic kidney disease, diabetes (such as type 2 diabetes), insulin resistance, non-alcoholic fatty liver disease, non-alcoholic steatohepatitis, pain, opioid addiction, obesity, cancer, inflammation (including chronic inflammatory diseases), autoimmune diseases, osteoporosis and intestinal diseases. Other diseases or conditions that may be ameliorated by the activation of AMPK include hyperinsulinemia and associated conditions, a condition/disorder where fibrosis plays a role, sexual dysfunction and neurodegenerative diseases.

The term “diabetes” (i.e. diabetes mellitus) will be understood by those skilled in the art to refer to both type 1 (insulin-dependent) diabetes and type 2 (insulin-independent) diabetes, both of which involve the malfunction of glucose homeostasis. The method of the invention is particularly suited for the treatment of diabetes, i.e. type 1 diabetes and/or type 2 diabetes , most particularly type 2 diabetes as is detailed in international patent application no. WO 2020/095010.

As well as being useful in the treatment of diabetes, the method of the invention is also suitable for treating diabetic kidney disease (i.e. diabetic nephropathy). “Diabetic kidney disease” refers to kidney damage caused by diabetes and is a serious complication of type 1 diabetes and type 2 diabetes. Diabetic kidney disease affects the kidneys' ability to remove waste products from blood to be excreted as urine, and can lead to kidney failure.

The method of the invention is also suitable for treating chronic kidney disease, including chronic kidney disease in the absence of type 2 diabetes. Chronic kidney disease is a condition characterised by a gradual loss of kidney function over time. Chronic kidney disease usually occurs as a result of one or more other diseases or conditions that affect the kidneys, such as high blood pressure, diabetes, high cholesterol, kidney infections, glomerulonephritis, polycystic kidney disease, obstruction of the urinary tract blockages in the flow of urine and long-term medication use.

The term “hyperinsulinemia or an associated condition” will be understood by those skilled in the art to include hyperinsulinemia, type 2 diabetes, glucose intolerance, insulin resistance, metabolic syndrome, dyslipidemia, hyperinsulinism in childhood, hypercholesterolemia, high blood pressure, obesity, fatty liver conditions, diabetic nephropathy, diabetic neuropathy, diabetic retinopathy, cardiovascular disease, atherosclerosis, cerebrovascular conditions such as stroke, systemic lupus erythematosus, neurodegenerative diseases such as Alzheimer's disease, and polycystic ovary syndrome. Other disease states include progressive renal disease such as chronic renal failure.

In particular, the method of the invention is suitable for the treatment of obesity associated with hyperinsulinemia and/or cardiovascular disease associated with hyperinsulinemia.

The method of the invention is also suitable for the treatment of cardiovascular disease, such as heart failure, wherein said cardiovascular disease is not associated with hyperinsulinemia. Similarly, the method of the invention is also suitable for use in the treatment of obesity which is not associated with hyperinsulinemia. For the avoidance of doubt, the treatment of obesity and/or cardiovascular disease (such as heart failure) where AMPK activation may be beneficial is included within the scope of the invention. In particular, the disease or disorder is heart failure, preferably heart failure with preserved ejection fraction (i.e. HFpEF).

The term “cancer”' will be understood by those skilled in the art to include one or more diseases in the class of disorders that is characterized by uncontrolled division of cells and the ability of these cells to invade other tissues, either by direct growth into adjacent tissue through invasion, proliferation or by implantation into distant sites by metastasis. By “proliferation” we include an increase in the number and/or size of cancer cells. By “metastasis” we mean the movement or migration (e.g. invasiveness) of cancer cells from a primary tumor site in the body of a subject to one or more other areas within the subject's body (where the cells can then form secondary tumors).

Thus, method of the invention is suitable for the treatment of any cancer type, including all tumors (non-solid and, preferably, solid tumors, such as carcinoma, adenoma, adenocarcinoma, blood cancer, irrespective of the organ). For example, the cancer cells may be selected from the group consisting of cancer cells of the breast, bile duct, brain, colon, stomach, reproductive organs, thyroid, hematopoietic system, lung and airways, skin, gallbladder, liver, nasopharynx, nerve cells, kidney, prostate, lymph glands and gastrointestinal tract. Preferably, the cancer is selected from the group consisting of colon cancer (including colorectal adenomas), breast cancer (e.g. postmenopausal breast cancer), endometrial cancer, cancers of the hematopoietic system (e.g. leukemia, lymphoma, etc.), thyroid cancer, kidney cancer, oesophageal adenocarcinoma, ovarian cancer, prostate cancer, pancreatic cancer, gallbladder cancer, liver cancer and cervical cancer. More preferably, the cancer is selected from the group consisting of colon, prostate and, particularly, breast cancer. Where the cancer is a non-solid tumor, it is preferably a hematopoietic tumor such as a leukemia (e.g. Acute Myelogenous Leukemia (AML), Chronic Myelogenous Leukemia (CML), Acute Lymphocytic Leukemia (ALL), or Chronic Lymphocytic Leukemia (CLL). Preferably the cancer cells are breast cancer cells.

A condition/disorder where fibrosis plays a role includes (but is not limited to) scar healing, keloids, scleroderma, pulmonary fibrosis (including idiopathic pulmonary fibrosis), nephrogenic systemic fibrosis, and cardiovascular fibrosis (including endomyocardial fibrosis), systemic sclerosis, liver cirrhosis, eye macular degeneration, retinal and vitreal retinopathy, Crohn's/inflammatory bowel disease, post-surgical scar tissue formation, radiation and chemotherapeutic-drug induced fibrosis, and cardiovascular fibrosis.

The method of invention is also be suitable for the treatment of sexual dysfunction (e.g. the treatment of erectile dysfunction). The method of invention may also be suitable for the treatment of inflammation.

Neurodegenerative diseases that may be mentioned include Alzheimer's disease, Parkinson's disease and Huntington's disease, amyotrophic lateral sclerosis, polyglutamine disorders, such as spinal and bulbar muscular atrophy (SBMA), dentatorubral and pallidoluysian atrophy (DRPLA), and a number of spinocerebellar ataxias (SCA).

The method of the invention is suitable for the treatment of a non-alcoholic fatty liver disease (NAFLD).

Non-alcoholic fatty liver disease (NAFLD) is defined by excessive fat accumulation in the form of triglycerides (steatosis) in the liver (designated as an accumulation of greater than 5% of hepatocytes histologically). It is the most common liver disorder in developed countries (for example, affecting around 30% of US adults) and most patients are asymptomatic. If left untreated, the condition may progressively worsen and may ultimately lead to cirrhosis of the liver. NAFLD is particularly prevalent in obese patients, with around 80% thought to have the disease.

NAFLD may be diagnosed wherein alcohol consumption of the patient is not considered to be a main causative factor. A typical threshold for diagnosing a fatty liver disease as “not alcohol related” is a daily consumption of less than 20 g for female subjects and less than 30 g for male subjects.

Particular diseases or conditions that are associated with NAFLD include metabolic conditions such as diabetes, hypertension, obesity, dyslipidemia, abetalipoproteinemia, glycogen storage diseases, Weber-Christian disease, acute fatty liver of pregnancy, and lipodystrophy. Other non-alcohol related factors related to fatty liver diseases include malnutrition, total parenteral nutrition, severe weight loss, refeeding syndrome, jejunoileal bypass, gastric bypass, polycystic ovary syndrome and diverticulosis.

Non-alcoholic steatohepatitis (NASH) is the most aggressive form of NAFLD, and is a condition in which excessive fat accumulation (steatosis) is accompanied by inflammation of the liver. If advanced, NASH can lead to the development of scar tissue in the liver (fibrosis) and, eventually, cirrhosis. As described above, compounds that activate AMPK have been found to be useful in the treatment of NAFLD and inflammation. It follows that the method of the invention is also useful in the treatment of NASH. Therefore, in a further embodiment, the treatment is of non-alcoholic steatohepatitis (NASH).

It has been shown that AMPK activator compounds (such as 4-chloro-N-[2-[(4-chlorophenyl)methyl]-3-oxo-1,2,4-thiadiazol-5-yl]benzamide (i.e. the compound of formula I)) are capable of treating pain (Das V, et al. Reg Anesth Pain Med 2019;0:1-5. doi:10.1136/rapm-2019-100839 and Das V, et al. Reg Anesth Pain Med 2019;0:1-6. doi:10.1136/rapm-2019-100651) and such compounds may be considered to be analgesics. It therefore follows that, since the method of the invention increases the bioavailability of a known AMPK activator compound, the methods of the invention may be suitable for the treatment of pain. In particular, the methods of the invention may be suitable for the treatment of patients with severe pain, chronic pain or useful in the management of pain after surgery.

Opioid-based therapies, such as opioid analgesics, are used to treat severe, chronic cancer pain, acute pain (e.g. during recovery from surgery and breakthrough pain) and their use is increasing in the management of chronic, non-malignant pain. However, the increasing use of opioid-based therapies to treat pain has resulted in an increase of opioid dependence (e.g. opioid addiction). By providing a known AMPK activator, the methods of the invention may be used to treat pain in place of an opioid-based therapy, as known by those skilled in the art. Accordingly, the method of the invention is suitable for treating opioid addiction.

Particular autoimmune diseases know to those skilled in the art include Crohn's/inflammatory bowel disease, systemic lupus erythematosus and type 1 diabetes.

Particular intestinal diseases that should be mentioned include Crohn's/inflammatory bowel disease and cancer of gastrointestinal tract.

The skilled person will understand that references to the “treatment” of a particular condition (or, similarly, to “treating” that condition) will take their normal meanings in the field of medicine. In particular, the terms may refer to achieving a reduction in the severity and/or frequency of occurrence of one or more clinical symptom associated with the condition, as judged by a physician attending a subject having or being susceptible to such symptoms.

The skilled person will understand that such treatment will be performed in a subject in need thereof. The need of a subject for such treatment may be assessed by those skilled the art using routine techniques. In the context of the present invention, a “subject in need” of the method of the invention includes a subject that is suffering from a disorder or condition ameliorated by the activation of AMPK. As used herein, the terms “disease” and “disorder” (and, similarly, the terms condition, illness, medical problem, and the like) may be used interchangeably.

Without wishing to be bound by theory, it is believed that the administration of a reduced dose of 4-chloro-N-[2-[(4-chlorophenyl)methyl]-3-oxo-1,2,4-thiadiazol-5-yl]benzamide to a human subject can still yield a clinically useful concentration of 4-chloro-N-[2-[(4-chlorophenyl)methyl]-3-oxo-1,2,4-thiadiazol-5-yl]benzamide in the systemic circulation when the active ingredient is administered in the form of the sodium salt of that compound. Repeated closing using the salt of the invention in the amount of from 200 to 1000 mg/day has been shown to result in a blood plasma concentration 4-chloro-N-[2-[(4-chlorophenyl)methyl]-3-oxo-1,2,4-thiadiazol-5-yl]benzamide that is approximately five-fold higher than the concentration obtained following repeated administration of the same amount of 4-chloro-N-[2-[(4-chlorophenyl)methyl]-3-oxo-1,2,4-thiadiazol-5-yl]benzamide in the free base form.

### Combination Therapies

The skilled person will understand that the method of the invention may comprise be combined with) further treatment(s) for the same condition.

In particular, when treating a disease or disorder that is ameliorated by activating AMPK, the salt of the invention may be administered in conjunction with one or more other (i.e. different) therapeutic agents that are useful in treating that disease or disorder.

Such combination treatments may involve the administration of the salt of the invention to the subject in conjunction (i.e. sequentially or simultaneously) with the different therapeutic agent in the same formulation, or preferably in a separate formulation. By “administration in conjunction with” (and similarly “administered in conjunction with”) we include that the respective active ingredients are administered, sequentially or simultaneously, as part of a medical intervention directed towards treatment of the relevant condition. By simultaneously, we mean that the salt of the invention and the different therapeutic agent are administered alongside one another, either in a single pharmaceutical dosage form comprising both active ingredients or in separate dosage forms administered at the same time.

Thus, in relation to the present invention, the term “administration in conjunction with” (and similarly “administered in conjunction with”) includes that the salt of the invention and the different therapeutic agent are administered either together, or sufficiently closely in time, to enable a beneficial effect for the patient that is greater, over the course of the treatment of the relevant condition, than if either agent is administered alone in the absence of the other component over the same course of treatment. Determination of whether a combination provides a greater beneficial effect in respect of, and over the course of, treatment of a particular condition will depend upon the condition to be treated, but may be achieved routinely by the skilled person.

Further, in the context of the present invention, the term “in conjunction with” includes that one or other of the two active ingredients may be administered (optionally repeatedly) prior to, after, and/or at the same time as, administration of the other. When used in this context, the terms “administered simultaneously” and “administered at the same time as” include instances in which the individual doses of the salt of the invention and the different therapeutic agent are administered within 6 hours, 3 hours, 2 hours, 1 hour, 45 minutes, 30 minutes, 20 minutes or 10 minutes) of each other.

Other therapeutic agents useful in treating a disease or disorder that is ameliorated by activating AMPK (such as heart failure, diabetic kidney disease, diabetes and the like, as described herein) will be well-known to those skilled in the art. Preferably the other therapeutic agent will be a sodium-glucose transport protein 2 (SGLT2) inhibitor, or a pharmaceutically acceptable salt, solvate or prodrug thereof such that the combination is useful for treating diseases such as type 2 diabetes. In a further embodiment, the method of the invention involves sequential or simultaneous administration of the sodium salt of 4-chloro-N-[2-[(4-chlorophenyl)methyl]-3-oxo-1,2,4-thiadiazol-5-yl]benzamide and the SGLT2 inhibitor.

The skilled person will understand that a sodium-glucose transport protein 2 inhibitor is a substance or agent that elicits a decrease in one or more functions of sodium-glucose transport protein 2, and by “decrease in the functions of sodium-glucose transport protein 2” we include the cessation of one or more functions of sodium-glucose transport protein 2, or a reduction in the rate of a particular function. A particular function that may be fully or partially inhibited is the ability of sodium-glucose transport protein 2 to act as a glucose transporter.

In particular embodiments, the sodium-glucose transport protein 2 inhibitor is a gliflozin. Gliflozins are a known class of small-molecule sodium-glucose transport protein 2 inhibitors. Hawley et al. (Diabetes, 2016, 65, 2784-2794) and Villani et al. (Molecular Metabolism, 2016, 5, 1048-1056) have recently discussed the possible mechanisms of action of certain gliflozins. Particular gliflozins which may be mentioned include dapagaliflozin, canagliflozin, empagliflozin, ipragliflozin, tofogliflozin, sergliflozin (such as sergliflozin etabonate), remogliflozin (such as remogliflozin etabonate), ertugliflozin and sotagliflozin. In further particular embodiments, the sodium-glucose transport protein 2 inhibitor is dapagliflozin.

In particular embodiments, the sodium-Glucose transport protein 2 inhibitor is a pharmaceutically acceptable salt of a gliflozin. For example, the further active ingredient may be a pharmaceutically acceptable salt of dapagliflozin, canagliflozin, empagliflozin, ipragliflozin, tofogliflozin, sergliflozin (such as sergliflozin etabonate), remogliflozin (such as remogliflozin etabonate), ertugliflozin or sotagliflozin.

In further embodiments, the sodium-glucose transport protein 2 inhibitor is a solvate of a gliflozin. For example, the further active ingredient may be a solvate of dapaaliflozin, canagliflozin, empagliflozin, ipraglifiozin, tofogliflozin, sergliflozin (such as sergliflozin etabonate), remogliflozin (such as remogliflozin etabonate), ertugliflozin or sotagliflozin.

In yet further embodiments, the sodium-glucose transport protein 2 inhibitor is a prodrug of a gliflozin. For example, the further active ingredient may be a prodrug of dapagliflozin, canagliflozin, empagliflozin, ipraglifiozin, tofogliflozin, sergliflozin (such as sergliflozin etabonate), remogliflozin (such as remogliflozin etabonate), ertugliflozin or sotaglifiozin.

The methods of the invention (and oral dosage forms used in such methods) disclosed herein may also have the advantage that the dose-efficient methods using the salt of the invention may be more efficacious than, be less toxic than, be longer acting than, be more potent than, produce fewer side effects than, be more easily absorbed than, and/or have a better pharmacokinetic profile (e.g. higher oral bioavailability and/or lower clearance) than over other therapies known in the prior art, whether for use in the above-stated indications or otherwise. In particular, methods of the invention may have the advantage that they are more efficacious and/or exhibit advantageous properties in vivo such as fewer side effects as a result of the dose-efficient characteristics of the salt of the invention.

### FIGURES

The following drawings are provided to illustrate various aspects of the present inventive concept and are not intended to limit the scope of the present invention unless specified herein.

FIG. 1 shows comparative results of oral pharmacokinetic studies (days 1 and 18) using 200, 400 and 800 mg doses of the salt of the invention.

FIG. 2 shows comparative results of oral pharmacokinetic studies (days 16 to 19 and 25) using 200, 400 and 800 mg doses of the salt of the invention.

FIG. 3 shows comparative results of oral pharmacokinetic studies (day 21) using 212.12 and 424.24 mg doses of the salt of the invention. These doses correspond to 200 mg and 400 mg, respectively, of 4-chloro-N-[2-[(4-chlorophenyl)methyl]-3-oxo-1,2,4-thiadiazol-5-yl]benzamide.

### EXAMPLES

### Abbreviations

AUC₀₋₂₄: Area under the plasma concentration-time curve from time zero to 24 days
AUCₗₐₛₜ: Area under the plasma concentration-time curve from time zero to last quantifiable concentration
AUCₛₛ: Area under the plasma concentration-time curve steady state
AUCₜ: Area under the plasma concentration-time curve from time zero to time t
AUCₜₐᵤ: Area under the plasma concentration-time curve during a dosage interval (τ); τ=tau
Cₘₐₓ: Peak plasma concentration
C_{trough}: Minimum plasma concentration
IMP: Investigational medicinal product
Tₘₐₓ: Time to reach the peak plasma concentration

The present invention will be further described by reference to the following examples, which are not intended to limit the scope of the invention.

### Example 1—Preparation of 4-Chloro-N-[2-[(4-Chlorophenyl)Methyl]-3-Oxo-1,2,4-Thiadiazol-5-Yl]Benzamide Sodium Salt

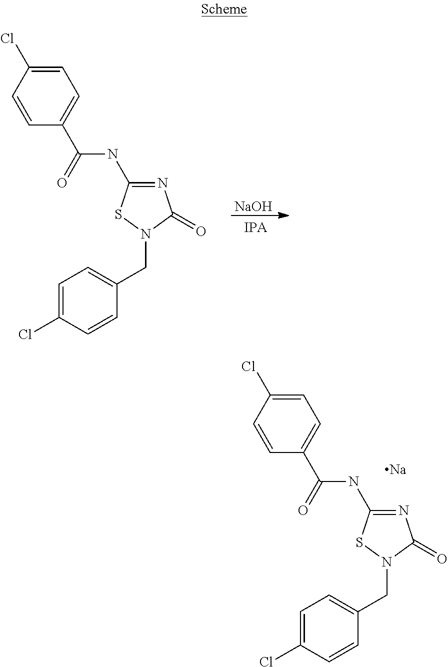

### Procedure:

To a suspension of 4-chloro-N-[2-[(4-chlorophenyl)methyl]-3-oxo-1,2,4-thiadiazol-5-yl]benzamide (100 g, 0.2629 mol) in isopropanol (1.0 L) was added slowly a solution of sodium hydroxide (11.56 g, 0.2891 mol) in water (100 mL) at 25±5° C. The mass was stirred for 3 h at 25±5° C. and cooled to 5±5° C. The mass was stirred for 3 h at 5±5° C. and filtered to collect the solids. The solids were washed with isopropanol (300 mL) and dried for 8 h under reduced pressure at 35±5° C. The dried solids were micronized twice using an air jet mill with 4.0 kg/cm² of primary pressure, 7.0 kg/cm² of secondary pressure and screw feeder with 8 RPM to isolate the desired sodium salt as white solid (50 g, 48%).

Repeated dry milling using a jet mill under argon at a primary pressure up to 4.0 kg/cm² and the secondary pressure up to 7.0 kg/cm² as used to give a particle size distribution with a D10 value of 0.3 μm, a D50 value of 1.9 μm and a D90 value of 7.1 μm (as determined by laser diffraction (Malvern Instrument, Mastersizer 3000)).

### Example 2—Pharmacokinetic Studies in Humans

The test material used in Example 2 was a sodium salt of 4-chloro-N-[2-[(4-chlorophenyl)methyl]-3-oxo-1,2,4-thiadiazol-5-yl]benzamide. This substance is referred to below as “the test material” and similar. The test material used in the study was synthesized and purified by Anthem Bioscience Pvt. Ltd. (Bangalore, India). The drug product containing the test material was produced by RISE (Södertälje, Sweden) for Betagenon AB (Umeå, Sweden)

### Methods

### Clinical Study Design

An open, randomised, parallel-group study in healthy volunteers (hereafter referred to as subjects) was conducted to evaluate the exposure of 4-chloro-N-[2-[(4-chlorophenyl)methyl]-3-oxo-1,2,4-thiadiazol-5-yl]benzamide after single and multiple dose administration of 3 dose levels of the sodium salt of 4-chloro-N-[2-[(4-chlorophenyl)methyl]-3-oxo-1,2,4-thiadiazol-5-yl]benzamide and to evaluate combination with dapagliflozin at steady state.

Main inclusion criteria: Healthy male subjects and healthy female subjects of non-childbearing potential, 18-65 years of age, with a body mass index (BMI)≥18.0 and ≤30.0 kg; m².

Main exclusion criteria: Subjects with a history of any clinically significant disease or disorder which, in the opinion of the Investigator, may either put the subject at risk, or influence the results or the subject's ability to participate in the study.

### Clinical Study Compound

A good-manufacturing practice (GMP) 1 kg batch of the test material was manufactured according to a method analogous to that in Example 1. The compound was provided in the form of a white to off-white crystalline powder with a D90 <8 μm, determined by laser diffraction (Malvern Instrument, Mastersizer 3000).

For the study, Vcaps enteric capsules, size 0, were individually filled with 200 mg of milled test material together with 2 mg of sodium docusate, 125 mg of mannitol and 6.5 mg of sodium lauryl sulfate.

Commercially available oral tablets of dapagliflozin, 10 mg, were provided.

### Clinical Methodology

Twenty-four (24) subjects were randomised (1:1:1) to treatment with either 200 mg (1 capsule of 200 mg), 400 mg (2 capsules of 200 mg) or, 800 mg (4 capsules of 200 mg) of the test material.

A screening visit (Visit 1) was performed within 28 days before randomisation and the start of IMP administration. The subjects were confined to the research clinic from the evening before Day 1 (Visit 2; Day—1). The subjects were randomised on Day 1 and allocated to one of three parallel dose groups of the IMP: 200, 400 or 800 mg once daily (1:1:1).

Pre-dose safety assessments as well as pre- and post-dose PK assessments were performed. The subjects left the clinic after the last PK-sample on Day 1 and returned for a 24 h PK sample and dosing on Day 2 (Visit 3).

Subjects self-administered the test material at home from Day 3 to Day 15. A telephone visit to check compliance, AE status and use of concomitant medications will be performed on Day 8 (Visit 4).

The subjects returned to the clinic on Days 16 and 17 (Visits 5 and 6), for PK sampling and AE assessment. Visit 6 continued in the evening of Day 17, when the subjects were confined to the clinic until Day 18. On Day 18, pre- and post-dose PK and safety assessments were performed. The subjects left the clinic after the last PK sample on Day 18 and returned for a 24 h PK sample and dosing on Day 19 (Visit 7).

On Day 19 (Visit 7) subjects began co-administration of the test material and dapagliflozin (Forxiga), 10 mg per day for 7 days (Day 19 to Day 25). The subjects returned to the clinic on Day 25 (Visit 8) for final dosing, PK and safety assessments. A final end-of-study visit (Visit 9) for follow-up of safety assessments was conducted 28 days (+14 days) after Visit 8 or after early withdrawal.

Venous blood samples (approximately 5 mL) for the determination of plasma concentrations of 4-chloro-N-[2-[(4-chlorophenyl)methyl]-3-oxo-1,2,4-thiadiazol-5-yl]benzamide after administration of the test material were collected through an indwelling venous catheter on Days 1, 2, 16 to 19 and 25.

The blood samples were collected in pre-labelled Li-Heparin tubes. All the collected blood samples were be centrifuged at 1500 G for 10 minutes to separate plasma within 60 minutes from the sample is drawn. The separated plasma from each blood sample was be divided into 2 aliquots in pre-labelled polypropylene cryotubes (A and B samples, approximately 750 μL in each tube) and frozen at <−70° C. On Day 25, the plasma was divided in to 3 aliquots of at least 500 μL (A and B samples for the test material, and 1 sample for potential future analysis of dapagliflozin).

Samples for determination of plasma concentrations of 4-chloro-N[2-[(4-chlorophenyl)methyl]-3-oxo-1,2,4-thiadiazol-5-yl]benzamide were analysed by Lablytica Life Science AB, Uppsala, Sweden by means of a validated LS-MS/MS method.

### Data Analysis

The data of plasma concentration to respective time points for the analyte (4-chloro-N-[2-[(4-chlorophenyl)methyl]-3-oxo-1,2,4-thiadiazol-5-yl]benzamide) were used for the pharmacokinetic analysis. Pharmacokinetic analysis was performed using non-compartmental analysis (NCA) module of Phoenix WinNonlin 8.1 software to determine the following pharmacokinetic parameters.

After single dose administration of test material:
AUC₀₋₂₄, Cₘₐₓ, Tₘₐₓ and dose proportionality (based on AUC₀₋₂₄ and Cₘₐₓ) (secondary endpoints).

After multiple dose administration of test material:
AUCₜₐᵤ and Cₘₐₓ, at steady state (primary endpoints)
AUCₜ, AUCₛₛ, Tₘₐₓ (secondary endpoints)
Dose proportionality after multiple doses, based on AUC at steady state (AUCₛₛ) and Cₘₐₓ (secondary endpoints)
C_{trough} pre-dose on Days 16 and 17 (secondary endpoint)
Accumulation ratio (secondary endpoint).

### Results

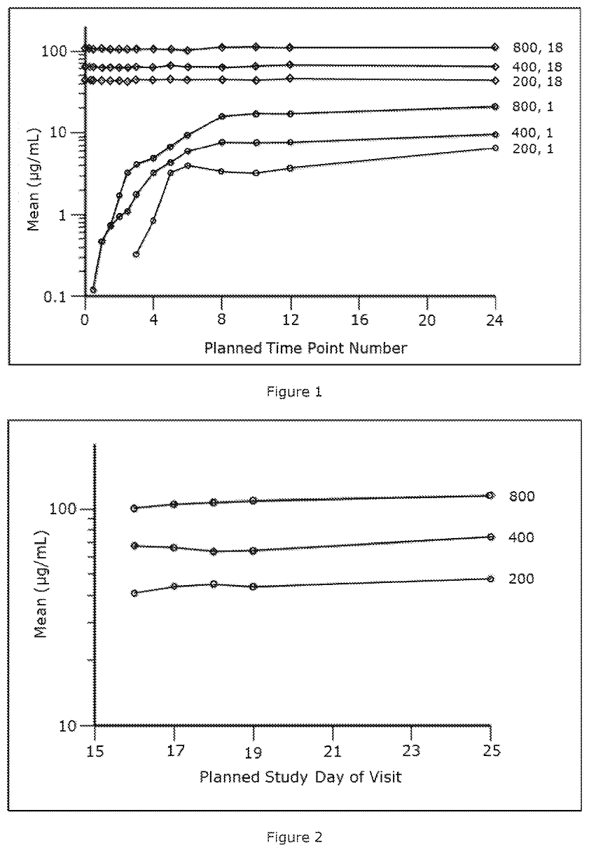

The results for the multiple dose oral pharmacokinetic study in humans are tabulated in Tables 1-4 below and are shown graphically in FIGS. 1 and 2.

The results show that there was substantial accumulation of 4-chloro-N-[2-[(4-chlorophenyl)methyl]-3-oxo-1,2,4-thiadiazol-5-yl]benzamide in the blood plasma after repeat dosing. Trough concentrations are similar on days 16 to 25, suggesting that steady state conditions (or at least close to steady state) have been established before day 18. At day 18, there is very little variation in the plasma concentration and a large increase in Cₘₐₓ compared to previous phase I and phase IIa pharmacokinetic studies performed with 4-chloro-N-[2-[(4-chlorophenyl)methyl]-3-oxo-1,2,4-thiadiazol-5-yl]benzamide in suspension.

In previous phase IIa studies involving patients with type-2 diabetes receiving 1000 mg/day of 4-chloro-N-[2-[(4-chlorophenyl)methyl]-3-oxo-1,2,4-thiadiazol-5-yl]benzamide in suspension, the mean Cₘₐₓ at day 28 was 55 μg/ml. This was an exploratory proof-of-concept randomised, parallel-group, double-blinded, placebo-controlled phase IIa 28-day study (TELLUS) conducted in 65 patients on Metformin for ≥3 months. TELLUS is listed in the EudraCT database protocol no. 2016-002183-13.

When compared to a standard suspension of the active ingredient, there was a surprising and substantial (up to five-fold) increase in the systemic exposure of 4-chloro-N-[2-[(4-chlorophenyl)methyl]-3-oxo-1,2,4-thiadiazol-5-yl]benzamide when 200 mg of the sodium salt of the active ingredient was administered to the subject.

TABLE 1
Mean Plasma Pharmacokinetic Parameters For the Analyte
(4-chloro-N-[2-[(4-chlorophenyl)methyl]-3-oxo-
1,2,4-thiadiazol-5-yl]benzamide) following administration
of the sodium salt of the active ingredient in capsule

| Dose | | Cₘₐₓ | Tₘₐₓ | AUCₗₐₛₜ |
|---|---|---|---|---|
| group | Sampling | (μg/mL) | (h) | (h*μg/mL) |
| 200 | Day 1 | 7.193 | 14.98 | 87.37 |
| | Day 18 | 49.86 | 8.025 | 1061 |
| 400 | Day 1 | 10.62 | 15.37 | 158.5 |
| | Day 18 | 70.77 | 8.31 | 1541 |
| 800 | Day 1 | 22.76 | 15.59 | 340.5 |
| | Day 18 | 118.5 | 11.48 | 2619 |

TABLE 2
Plasma Pharmacokinetic Parameters For the 2000 mg Dose Group

| | AUClast | AUC_TAU | CLss_F | Tmax | Cmax | Tlast | Clast | Cmax D | AUClast_D | AUC_TAU_D |
|---|---|---|---|---|---|---|---|---|---|---|
| SUBJECT | (h*μg/ml) | (h*μg/ml) | (ml/h) | (h) | (μg/ml) | (h) | (μg/ml) | (μg/ml/mg) | (h*μg/ml/mg) | (h*μg/ml/mg) |
| 1 | 800 | 802 | 249 | 8 | 40.2 | 23.9 | 33.3 | 0.201 | 4 | 4.01 |
| 4 | 947 | 950 | 210 | 12 | 46.2 | 23.9 | 36.7 | 0.231 | 4.74 | 4.75 |
| 5 | 772 | 774 | 259 | 5 | 34.6 | 23.9 | 30.4 | 0.173 | 3.86 | 3.87 |
| 7 | 1010 | 1010 | 198 | 5 | 49.9 | 23.9 | 40.7 | 0.25 | 5.04 | 5.06 |
| 8 | 1130 | 1130 | 176 | 0.267 | 52.1 | 23.9 | 47 | 0.261 | 5.65 | 5.67 |
| 13 | 1400 | | | 23.9 | 62 | 23.9 | 62 | 0.31 | 6.98 | |
| 15 | 838 | 841 | 238 | 0 | 42 | 23.9 | 33.5 | 0.21 | 4.19 | 4.2 |
| 18 | 1600 | 1600 | 125 | 10 | 71.9 | 23.9 | 68 | 0.36 | 7.98 | 8.01 |
| N | 8 | 7 | 7 | 8 | 8 | 8 | 8 | 8 | 8 | 7 |
| Mean | 1061 | 1016 | 207.9 | 8.025 | 49.86 | 23.92 | 43.95 | 0.2493 | 5.305 | 5.082 |
| SD | 298 | 288 | 46.9 | 7.71 | 12.2 | 0.00863 | 14.1 | 0.0609 | 1.49 | 1.44 |
| CV % | 28.1 | 28.3 | 22.5 | 96 | 24.4 | 0 | 32 | 24.4 | 28.1 | 28.3 |
| Min | 772 | 774 | 125 | 0 | 34.6 | 23.9 | 30.4 | 0.173 | 3.86 | 3.87 |
| Median | 977.7 | 950.3 | 210.5 | 6.5 | 48.05 | 23.92 | 38.7 | 0.2403 | 4.888 | 4.752 |
| Max | 1600 | 1600 | 259 | 23.9 | 71.9 | 23.9 | 68 | 0.36 | 7.98 | 8.01 |
| Geometric | 1028 | 986.8 | 202.7 | | 48.63 | 23.92 | 42.19 | 0.2432 | 5.139 | 4.934 |
| Mean | | | | | | | | | | |
| Geometric | 26.9 | 25.7 | 25.7 | | 24 | 0 | 30.6 | 24 | 26.9 | 25.7 |
| CV % | | | | | | | | | | |

TABLE 3
Plasma Pharmacokinetic Parameters For the 400 mg Dose Group

| | AUClast | AUC_TAU | CLss_F | Tmax | Cmax | Tlast | Clast | Cmax_D | AUClast_D | AUC_TAU_D |
|---|---|---|---|---|---|---|---|---|---|---|
| SUBJECT | (h*μg/ml) | (h*μg/ml) | (ml/h) | (h) | (μg/ml) | (h) | (μg/ml) | (μg/ml/mg) | (h*μg/ml/mg) | (h*μg/ml/mg) |
| 2 | 1480 | 1480 | 270 | 12 | 66.1 | 24 | 60.9 | 0.165 | 3.7 | 3.71 |
| 3 | 1320 | 1320 | 303 | 5 | 64.2 | 23.9 | 49.2 | 0.161 | 3.29 | 3.3 |
| 9 | 2090 | 2100 | 191 | 0.25 | 99.1 | 23.9 | 86.9 | 0.248 | 5.23 | 5.24 |
| 12 | 1650 | | | 23.9 | 72.9 | 23.9 | 72.9 | 0.182 | 4.13 | |
| 14 | 1210 | 1210 | 330 | 12 | 56.2 | 23.9 | 54.7 | 0.141 | 3.02 | 3.03 |
| 17 | 1290 | 1300 | 308 | 2 | 59.8 | 23.9 | 50.4 | 0.15 | 3.23 | 3.24 |
| 21 | 1740 | 1750 | 229 | 3 | 77.1 | 23.9 | 73.2 | 0.193 | 4.36 | 4.37 |
| N | 7 | 6 | 6 | 7 | 7 | 7 | 7 | 7 | 7 | 6 |
| Mean | 1541 | 1526 | 271.8 | 8.31 | 70.77 | 23.93 | 64.03 | 0.1769 | 3.852 | 3.816 |
| SD | 311 | 338 | 53.2 | 8.31 | 14.4 | 0.0236 | 14.1 | 0.036 | 0.778 | 0.845 |
| CV % | 20.2 | 22.1 | 19.6 | 100.1 | 20.4 | 0.1 | 22 | 20.4 | 20.2 | 22.1 |
| Min | 1210 | 1210 | 191 | 0.25 | 56.2 | 23.9 | 49.2 | 0.141 | 3.02 | 3.03 |
| Median | 1481 | 1402 | 286.3 | 5 | 66.1 | 23.93 | 60.9 | 0.1653 | 3.703 | 3.504 |
| Max | 2090 | 2100 | 330 | 23.9 | 99.1 | 24 | 86.9 | 0.248 | 5.23 | 5.24 |
| Geometric | 1515 | 1498 | 267.1 | 4.269 | 69.64 | 23.93 | 62.75 | 0.1741 | 3.788 | 3.744 |
| Mean | | | | | | | | | | |
| Geometric | 19.7 | 21.2 | 21.2 | 301.9 | 19.1 | 0.1 | 21.8 | 19.1 | 19.7 | 21.2 |
| CV % | | | | | | | | | | |

TABLE 4
Plasma Pharmacokinetic Parameters For the 800 mg Dose Group

| | AUClast | AUC_TAU | CLss_F | Tmax | Cmax | Tlast | Clast | Cmax_D | AUClast_D | AUC_TAU_D |
|---|---|---|---|---|---|---|---|---|---|---|
| SUBJECT | (h*μg/ml) | (h*μg/ml) | (ml/h) | (h) | (μg/ml) | (h) | (μg/ml) | (μg/ml/mg) | (h*μg/ml/mg) | (h*μg/ml/mg) |
| 6 | 2980 | | | 10 | 130 | 23.9 | 127 | 0.163 | 3.73 | |
| 10 | 2720 | 2730 | 293 | 6 | 126 | 23.9 | 110 | 0.158 | 3.4 | 3.42 |
| 11 | 2580 | | | 23.9 | 117 | 23.9 | 117 | 0.146 | 3.22 | |
| 16 | 2860 | 2870 | 279 | 10 | 128 | 23.9 | 115 | 0.16 | 3.58 | 3.59 |
| 19 | 1800 | 1810 | 442 | 8 | 87.3 | 23.9 | 73.7 | 0.109 | 2.25 | 2.26 |
| 20 | 2490 | 2500 | 321 | 9.98 | 118 | 23.9 | 98 | 0.148 | 3.11 | 3.12 |
| 22 | 2900 | 2910 | 275 | 0 | 127 | 23.9 | 120 | 0.159 | 3.63 | 3.64 |
| 23 | 2620 | | | 23.9 | 115 | 23.9 | 115 | 0 | 144 | 3.27 |
| N | 8 | 5 | 5 | 8 | 8 | 8 | 8 | 8 | 8 | 5 |
| Mean | 2619 | 2564 | 321.8 | 11.48 | 118.5 | 23.92 | 109.5 | 0.1482 | 3.274 | 3.205 |
| SD | 372 | 452 | 69.7 | 8.37 | 13.8 | 0.00772 | 16.7 | 0.0173 | 0.465 | 0.565 |
| CV % | 14.2 | 17.6 | 21.6 | 72.9 | 11. | 0 | 15.2 | 11.7 | 14.2 | 17.6 |
| Min | 1800 | 1810 | 275 | 0 | 87.3 | 23.9 | 73.7 | 0.109 | 2.25 | 2.26 |
| Median | 2670 | 2732 | 292.8 | 9.992 | 122 | 23.92 | 115 | 0.1525 | 3.337 | 3.416 |
| Max | 2980 | 2910 | 442 | 23.9 | 130 | 23.9 | 127 | 0.163 | 3.73 | 3.64 |
| Geometric | 2592 | 2527 | 316.5 | | 117.7 | 23.92 | 108.2 | 0.1472 | 3.24 | 3.159 |
| Mean | | | | | | | | | | |
| Geometric | 16.1 | 19.8 | 19.8 | | 13 | 0 | 17.3 | 13 | 16.1 | 19.8 |
| CV % | | | | | | | | | | |

### Example 3—Pharmacokinetic Studies in Humans Using a New Tablet Formulation of O304 Na Salt

The test material used in Example 3 was a sodium salt of 4-chloro-N-[2-[(4-chlorophenyl)methyl]-3-oxo-1,2,4-thiadiazol-5-yl]benzamide. The free base form of this substance (i.e. 4-chloro-N-[2-[(4-chlorophenyl)methyl]-3-oxo-1,2,4-thiadiazol-5-yl]benzamide) is referred to below as “the test substance”, IMP and similar. The drug substance in the test material used in the study was synthesized by Anthem BioSciences (Bangalore, India) and the tablets (drug product) were produced by Recipharm Pharmaservices Pvt. Ltd. (Bangalore, India) for Betagenon Bio AB (Umeå, Sweden).

### Methods

### Clinical Study Design

An open, randomised, parallel-group study was conducted to evaluate the exposure and safety of 4-chloro-N-[2-[(4-chlorophenyl)methyl]-3-oxo-1,2,4-thiadiazol-5-yl]benzamide sodium salt tablets in doses of 212.12 mg and 424.24 mg (equivalent to 200 mg and 400 mg doses of 4-chloro-N-[2-[(4-chlorophenyl)methyl]-3-oxo-1,2,4-thiadiazol-5-yl]benzamide) at steady state in healthy volunteers (hereafter referred to as subjects).

Main inclusion criteria: Healthy male subjects and healthy female subjects of non-childbearing potential, 18-65 years of age, with a body mass index (BMI) ≥18.0 and ≤30.0 kg/m².

Main exclusion criteria: Subjects with a history of any clinically significant disease or disorder which, in the opinion of the Investigator, may either put the subject at risk, or influence the results or the subject's ability to participate in the study.

### Clinical Study Compound

Each “400 mg” tablet contained 424.24 mg of a sodium salt of 4-chloro-N-[2-[(4-chlorophenyl)methyl]-3-oxo-1,2,4-thiadiazol-5-yl]benzamide (Compound of formula II) which corresponds to 400 mg of the parent compound 4-chloro-N-[2-[(4-chlorophenyl)methyl]-3-oxo-1,2,4-thiadiazol-5-yl]benzamide.

The formulation details of the “400 mg” tablet are shown below in Table 5.

TABLE 5
Drug product composition of the “400” mg tablet

| | Content | | | Reference |
|---|---|---|---|---|
| Component | per tablet | | Function | to standard |
| Compound of formula II | 424.24 | mg | API | — |
| Microcrystalline | 206.59 | mg | Filler/Binder | Ph. Eur. |
| cellulose | | | | |
| Lactose monohydrate | 413.17 | mg | Filler | Ph. Eur. |
| Sodium starch glycolate | 90.00 | mg | Disintegrant | Ph. Eur. |
| Colloidal silicon dioxide | 30.00 | mg | Glidant | Ph. Eur. |
| Magnesium stearate | 36.00 | mg | Lubricant | Ph. Eur. |
| Total | 1200.00 | mg | | |

Each tablet had a score line, which made it possible to split the tablet into 2 pieces for administration of the lower dose of 200 mg of 4-chloro-N-[2-[(4-chlorophenyl)methyl]-3-oxo-1,2,4-thiadiazol-5-yl]benzamide.

### Clinical Methodology

Twenty (20) subjects were randomised (1:1) to treatment with either 200 mg (1 tablet of 200 mg) or 400 mg (1 tablet of 400 mg) of the test substance.

A screening visit (Visit 1) was performed within 28 days before randomisation and the start of IMP administration. The subjects arrived at the research clinic on Day 1 (Visit 2), randomised, and received the first administration of the test substance (200 mg or 400 mg as randomised).

The subjects returned to the clinic in the morning of Day 2 (Visit 3) for pre-dose PK sampling and subsequent administration of the test substance.

Before leaving the clinic, the subjects were provided with the test substance for 18 days once daily self-administration at home (Day 3 to Day 20).

In addition, the subjects were instructed how to use an electronic diary in which their daily intake of the test substance was registered. Subjects were contacted by phone on Day 11±1 (Visit 4) for a check-up of adverse events (AEs), use of concomitant medications and IMP accountability. Site personnel contacted subjects who do not regularly register IMP intake in the electronic diary.

After 18 days of home-based self-administration of the test substance, the subjects returned to the clinic in the morning of Day 21 (Visit 5) for pre-dose PK sampling and subsequent administration of the last dose of the test substance. The subjects remained at the clinic for at least 8 hours post-dose for safety assessments and additional PK sampling. In the morning of Day 22 (Visit 6), the subjects came for a last visit to the clinic for a final PK sample 24 hours after the last dose.

A final telephone-based end-of-study visit (Visit 7) will took place approximately 2 weeks after the last dose, on Day 35±3 days.

Venous blood samples (approximately 5 mL) for the determination of plasma concentrations of 4-chloro-N-[2-[(4-chlorophenyl)methyl]-3-oxo-1,2,4-thiadiazol-5-yl]benzamide after administration of the test substance were collected through an indwelling venous catheter on Days 2, 21 and 22.

The blood samples were collected in pre-labelled Li-Heparin tubes. All the collected blood samples were be centrifuged at 1500 G for 10 minutes to separate plasma within 60 minutes from the sample is drawn. The separated plasma from each blood sample was be divided into 2 aliquots in pre-labelled polypropylene cryotubes (A and B samples, approximately 750 μL in each tube) and frozen at <−70° C.

Samples for determination of plasma concentrations of 4-chloro-N-[2-[(4-chlorophenyl)methyl]-3-oxo-1,2,4-thiadiazol-5-yl]benzamide were analysed by Lablytica Life Science AB, Uppsala, Sweden by means of a validated LS-MS/MS method.

### Data Analysis

The data of plasma concentration to respective time points for the analyte (4-chloro-N-[2-[(4-chlorophenyl)methyl]-3-oxo-1,2,4-thiadiazol-5-yl]benzamide) were used for the pharmacokinetic analysis. Pharmacokinetic analysis was performed using non-compartmental analysis (NCA) module of Phoenix WinNonlin 8.1 software to determine the following pharmacokinetic parameters after multiple dose administration of test substance.

Primary endpoint:
Cₘₐₓ, AUC₀₋ₗₐₛₜ, Tₘₐₓ at steady state Day 21 in each dose group.
   Secondary endpoints:
Accumulation ratio based on plasma concentrations 24 hours after first and last dose.
Dose normalised Cₘₐₓ and AUC₀₋ₗₐₛₜ at steady state Day 21.

### Results

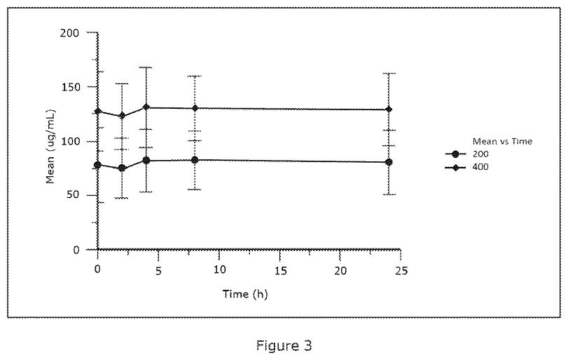

The results for the multiple dose oral pharmacokinetic study in humans are tabulated in Tables 6-9 below and are shown graphically in FIG. 3.

The results show again that there was substantial accumulation of 4-chloro-N-[2-[(4-chlorophenyl)methyl]-3-oxo-1,2,4-thiadiazol-5-yl]benzamide in the blood plasma after repeat dosing.

Moreover, there was a surprising and substantial (approximately seven-fold) increase in the systemic accumulation of 4-chloro-N-[2-[(4-chlorophenyl)methyl]-3-oxo-1,2,4-thiadiazol-5-yl]benzamide from Day 1 to Day 21 when 212 mg of the sodium salt of the active ingredient in tablet form was administered to the subject. An approximately five-fold increase in the systemic accumulation of 4-chloro-N-[2-[(4-chlorophenyl)methyl]-3-oxo-1,2,4-thiadiazol-5-yl]benzamide from Day 1 to Day 21 was observed for subjects administered with 424 mg of the sodium salt of the active ingredient in tablet form.

When compared to capsules containing 200 mg of the sodium salt of active ingredient, there was a significant (up to two-fold) increase in the systemic exposure of 4-chloro-N-[2-[(4-chlorophenyl)methyl]-3-oxo-1,2,4-thiadiazol-5-yl]benzamide when 212 mg of the sodium salt of the active ingredient in tablet form was administered to the subject.

Further, tablets and capsules containing the sodium salt of the active ingredient both provided surprisingly higher dose-normalized plasma exposure of 4-chloro-N-[2-[(4-chlorophenyl)methyl]-3-oxo-1,2,4-thiadiazol-5-yl]benzamide compared to multiple administered doses of the free base of the active ingredient in suspension.

TABLE 6
Comparison of Cₗₐₛₜ for the Analyte (4-chloro-N-[2[(4-
chlorophenyl)methyl]-3-oxo-1,2,4-thiadiazol-5-yl]benzamide)
at 24 hours between First and Last Doses for the
200 mg and 400 mg Dose Groups

| Dose | Time | | Mean | SD | Min | Median | Max |
|---|---|---|---|---|---|---|---|
| (mg) | (h) | Day | (μg/ml) | (μg/ml) | (μg/ml) | (μg/ml) | (μg/ml) |
| 200 | 24 | 1 | 11.37 | 3.57 | 7.01 | 11.20 | 17.8 |
| | | 21 | 80.42 | 29.56 | 51.7 | 76.90 | 144 |
| 400 | 24 | 1 | 24.94 | 5.80 | 19.5 | 21.50 | 34.9 |
| | | 21 | 129.0 | 33.23 | 96.7 | 116.0 | 201 |

TABLE 7
Day 21 Plasma Pharmacokinetic Parameters
for the 200 mg Dose Group

| | Tₘₐₓ | Cₘₐₓ | AUCₗₐₛₜ | Tₗₐₛₜ | Cₗₐₛₜ |
|---|---|---|---|---|---|
| SUBJECT | (h) | (μg/ml) | (h*μg/ml) | (h) | (μg/ml) |
| 102 | 4.00 | 77.1 | 1680 | 24.0 | 65.1 |
| 105 | 24.0 | 81.3 | 1870 | 24.0 | 81.3 |
| 106 | 4.00 | 58.6 | 1340 | 24.0 | 53.4 |
| 107 | 8.00 | 90.1 | 1990 | 24.0 | 80.1 |
| 108 | 24.0 | 109 | 2550 | 24.0 | 109 |
| 109 | 0.00 | 159 | 3460 | 24.0 | 144 |
| 111 | 2.00 | 57.4 | 1290 | 24.0 | 51.7 |
| 117 | 24.0 | 76.9 | 1720 | 24.0 | 76.9 |
| 119 | 8.00 | 71.7 | 1600 | 24.1 | 62.3 |
| N | 9 | 9 | 9 | 9 | 9 |
| Mean | 10.89 | 86.79 | 1943 | 24.01 | 80.42 |
| SD | 10.2 | 31.3 | 679 | 0.0400 | 29.6 |
| CV % | 93.3 | 36.0 | 34.9 | 0.2 | 36.8 |
| Min | 0.00 | 57.4 | 1290 | 24.0 | 51.7 |
| Median | 8.00 | 77.1 | 1720 | 24.0 | 76.9 |
| Max | 24.0 | 159 | 3460 | 24.1 | 144 |

TABLE 8
Day 21 Plasma Pharmacokinetic Parameters
for the 400 mg Dose Group

| | Tmax | Cmax | AUClast | Tlast | Clast |
|---|---|---|---|---|---|
| SUBJECT | (h) | (μg/ml) | (h*μg/ml) | (h) | (μg/ml) |
| 101 | 4.00 | 124 | 2800 | 24.0 | 111 |
| 103 | 24.0 | 106 | 2450 | 24.0 | 106 |
| 104 | 4.00 | 105 | 2460 | 24.0 | 100 |
| 110 | 4.02 | 132 | 2930 | 24.0 | 116 |
| 112 | 24.4 | 201 | 4790 | 24.4 | 201 |
| 113 | 4.00 | 169 | 3600 | 24.0 | 151 |
| 114 | 8.02 | 106 | 2450 | 24.1 | 96.7 |
| 115 | 24.1 | 135 | 2950 | 24.1 | 135 |
| 118 | 4.00 | 168 | 3550 | 24.0 | 144 |
| N | 9 | 9 | 9 | 9 | 9 |
| Mean | 11.17 | 138.4 | 3109 | 24.07 | 129.0 |
| SD | 9.83 | 33.9 | 768 | 0.134 | 33.2 |
| CV % | 88.0 | 24.5 | 24.7 | 0.6 | 25.8 |
| Min | 4.00 | 105 | 2450 | 24.0 | 96.7 |
| Median | 4.02 | 132 | 2930 | 24.0 | 116 |
| Max | 24.4 | 201 | 4790 | 24.4 | 201 |

TABLE 9
Comparison of Pharmacokinetic Parameters for the Test Formulations used in Examples 2 and 3 and a Multiple Administered
Dose (MAD) of 4-chloro-N-[2-[(4-chlorophenyl)methyl]-3-oxo-1,2,4-thiadiazol-5-yl]benzamide free base in a Suspension

| | | | Equivalent | | Mean | Mean | | Mean | Mean | |
|---|---|---|---|---|---|---|---|---|---|---|
| | | | Dose of API | | Cₘₐₓ | Cₘₐₓ, ss | Mean | Cₘₐₓ | Cₘₐₓ, ss | Mean |
| Test | Form of | Dose of | as free base | | Day 1 | QD | AUCₜₐᵤ | Day 1/Dose | QD/Dose | AUC/Dose |
| Formulation | API | API (mg) | (mg) | Pop | (μg/ml) | (μg/ml) | (h*μg/ml) | (h*μg/ml) | (h*μg/ml) | (h*μg/ml) |
| MAD1 | Free | 300/4 d | 300/4 d | HV | 4.5 | — | 736 | — | — | 2.45 |
| Suspension | base | | | | | | | | | |
| MAD2 | Free | 1200/4 d | 1200/4 d | HV | 10.8 | — | 2371 | — | — | 1.98 |
| Suspension | base | | | | | | | | | |
| MAD3 | Free | 2400/4 d | 2400/4 d | HV | 13.3 | — | 2388 | — | — | 1.06 |
| Suspension | base | | | | | | | | | |
| MAD4 | Free | 2400/4 d | 2400/4 d | Patients | 16.2 | — | 2639 | — | — | 1.09 |
| Suspension | base | | | | | | | | | |
| MAD5 | Free | 800 QD | 800 QD | HV | 7.6 | 48.3 | 1023 | — | 0.060 | 1.28 |
| Suspension | base | | | | | | | | | |
| Example 2 | Sodium | 200 QD | 189 QD | HV | 7.2 | 49.9 | 1016 | 0.036 | 0.249 | 5.08 |
| (capsule) | salt | | | | | | | | | |
| Example 2 | Sodium | 400 QD | 377 QD | HV | 10.6 | 70.8 | 1526 | 0.027 | 0.177 | 3.82 |
| (capsule) | salt | | | | | | | | | |
| Example 2 | Sodium | 800 QD | 754 QD | HV | 22.8 | 118.5 | 2564 | 0.029 | 0.145 | 3.21 |
| (capsule) | salt | | | | | | | | | |
| Example 3 | Sodium | 212 QD | 200 QD | HV | — | 86.8 | 1942 | — | 0.43 | 9.71 |
| (tablet) | salt | | | | | | | | | |
| Example 3 | Sodium | 424 QD | 400 QD | HV | — | 138.4 | 3098 | — | 0.35 | 7.74 |
| (tablet) | salt | | | | | | | | | |

### REFERENCES

1. Hardie D. G, AMPK-activated protein kinase-an energy sensor that regulates all aspects of cell function. Genes Dev. 25, 1895-1908, 2011.

2. Schulz E, et al., When Metabolism Rules Perfusion, AMPK-Mediated Endothelial Nitric Oxide Synthase Activation, Circulation Research. 104, 422-424, 2009.

3. Hardie D. G, et. al., AMP-activated protein kinase: A Target for Drugs both Ancient and Modern. Chemistry & Biology 19, 1222-1236, 2012.

4. Myers R. W, et al., Systemic pan-AMPK activator MK-8722 improves glucose homeostasis but induces cardiac hypertrophy. Science 357, 507-511, 2017.

5. Cokorinos E. C, et al., Activation of Skeletal Muscle AMPK Promotes Glucose Disposal and Glucose Lowering in Non-human Primates and Mice. Cell Metab. 2017;25(5):1147-1159, 2017

6. Steneberg P, et al., PAN-AMPK activator O304 improves glucose homeostasis and microvascular perfusion in mice and type 2 diabetes patients. JCI Insight. 3(12):e99114, 2018

7. National Cancer Institute Cancer Therapy Evaluation Program. Common terminology criteria for adverse events, CTCAE v5.0 (2017).

8. World Medical Association, WMA Declaration of Helsinki—Ethical principles for medical research involving human subjects [website], https://www.wma.net/policiespost/wma-declaration-of-helsinki-ethical-principles-for-medical-research-involvinghuman-subjects/, (accessed 16 Apr. 2020)

1. A method of activating 5′ adenosine monophosphate-activated protein kinase (AMPK) comprising administering from about 200 to about 1000 mg/day of a sodium salt of 4-chloro-N-[2-[(4-chlorophenyl)methyl]-3-oxo-1,2,4-thiadiazol-5-yl]benzamide in a pharmaceutical dosage form to a human subject.

2. The method according to claim 1, wherein from about 200 to about 800 mg/day or from about 200 to about 400 mg/day of the sodium salt of 4-chloro-N-[2-[(4-chlorophenyl)methyl]-3-oxo-1,2,4-thiadiazol-5-yl]benzamide is administered to the human subject.

3. The method according to claim 1 or claim 2, wherein the sodium salt of 4-chloro-N-[2-[(4-chlorophenyl)methyl]-3-oxo-1,2,4-thiadiazol-5-yl]benzamide is administered daily to the human subject and results in a peak blood plasma concentration of 4-chloro-N-[2-[(4-chlorophenyl)methyl]-3-oxo-1,2,4-thiadiazol-5-yl]benzamide of at least 40, 50, 60, 70, 80, 90, 100, 110, 120 or 130 μg/mL.

4. The method according to claim 3, wherein the peak blood plasma concentration is reached after achieving a steady state concentration.

5. The method according to claim 3, wherein the peak blood plasma concentration is reached after 15, 16, 17 or 18 days.

6. The method according to anyone of the preceding claims, wherein the sodium salt of 4-chloro-N-[2-[(4-chlorophenyl)methyl]-3-oxo-1,2,4-thiadiazol-5-yl]benzamide is provided in the form of particles having a particle size distribution defined by a D90 of less than about 10 μm.

7. The method according to any preceding claim, wherein the method activates 5′ adenosine monophosphate-activated protein kinase (AMPK) and thereby treats heart failure.

8. The method according to any one of claims 1 to 6, wherein the method activates 5′ adenosine monophosphate-activated protein kinase (AMPK) and thereby treats diabetic kidney disease.

9. The method according to any one of claims 1 to 6, wherein the method activates 5′ adenosine monophosphate-activated protein kinase (AMPK) and thereby treats diabetes.

10. The method according to any one of claims 1 to 9, further comprising administering a sodium-glucose transport protein 2 (SGLT2) inhibitor, or a pharmaceutically acceptable salt, solvate or prodrug thereof to the human subject.

11. The method according to claim 10, wherein the sodium salt of 4-chloro-N-[2-[(4-chlorophenyl)methyl]-3-oxo-1,2,4-thiadiazol-5-yl]benzamide and the SGLT2 inhibitor are administered sequentially or simultaneously to the human subject.

12. The method according to claim 10 or claim 11, wherein the SGLT2 inhibitor is dapagliflozin, canagliflozin, empagliflozin, ipragliflozin, tofogliflozin, serglifiozin etabonate, remogliflozin etabonate, ertugliflozin or sotagliflozin.

13. The method according to any one of the preceding claims, wherein the pharmaceutical dosage form comprises an enteric coating, preferably wherein the enteric coating comprises beeswax, shellac, an alkylcellulose polymer resin, an acrylic polymer resin, cellulose acetate phthalate or polyvinyl acetate phthalate.

14. The method according to any one of the preceding claims, wherein the pharmaceutical dosage form comprising the sodium salt of 4-chloro-N-[2-[(4-chlorophenyl)methyl]-3-oxo-1,2,4-thiadiazol-5-yl]benzamide further comprises at least one pharmaceutically acceptable excipient selected from the group consisting of a lubricant, a binder, a filler, a surfactant, a diluent, an anti-adherent, a coating, a flavouring, a colourant, a glidant, a preservative, a sweetener, a disintegrant, an adsorbent, a buffering agent, an antioxidant, a chelating agent, a dissolution enhancer, a dissolution retardant and a wetting agent.

15. The method according to any one of the preceding claims, wherein the pharmaceutical dosage form is a capsule or a tablet.

16. An oral pharmaceutical dosage form comprising from about 200 to about 1000 mg of a sodium salt of 4-chloro-N-[2-[(4-chlorophenyl)methyl]-3-oxo-1,2,4-thiadiazol-5-yl]benzamide.

17. The oral pharmaceutical dosage form according to claim 16, wherein the pharmaceutical dosage form comprises from about 200 to about 800 mg or from about 200 to about 400 mg of the sodium salt of 4-chloro-N-[2-[(4-chlorophenyl)methyl]-3-oxo-1,2,4-thiadiazol-5-yl]benzamide.

18. The oral pharmaceutical dosage form according to claim 16 or claim 17, wherein the pharmaceutical dosage form comprises an enteric coating, preferably wherein the enteric coating comprises beeswax, shellac, an alkylcellulose polymer resin, an acrylic polymer resin, cellulose acetate phthalate or polyvinyl acetate phthalate.

19. The oral pharmaceutical dosage form according to any one of claims 16 to 18, wherein the pharmaceutical dosage form further comprises at least one pharmaceutically acceptable excipient selected from the group consisting of a lubricant, a binder, a filler, a surfactant, a diluent, an anti-adherent, a coating, a flavouring, a colourant, a glidant, a preservative, a sweetener, a disintegrant, an adsorbent, a buffering agent, an antioxidant, a chelating agent, a dissolution enhancer, a dissolution retardant and a wetting agent.

20. The oral pharmaceutical dosage form according to any one of claims 16 to 19, wherein the pharmaceutical dosage form is a capsule or a tablet.

21. The oral pharmaceutical dosage form according to any one of claims 16 to 20, wherein the sodium salt of 4-chloro-N-[2-[(4-chlorophenyl)methyl]-3-oxo-1,2,4-thiadiazol-5-yl]benzamide is provided in the form of particles having a particle size distribution defined by a D90 of less than about 10 μm.

22. Use of an oral pharmaceutical dosage form according to any one of claims 16 to 21 in the manufacture of a medicament for the treatment of a disease or disorder by activating AMPK, wherein from about 200 to about 1000 mg/day of the sodium salt of 4-chloro-N-[2-[(4-chlorophenyl)methyl]-3-oxo-1,2,4-thiadiazol-5-yl]benzamide is administered to a human subject.
